(19) United States
(12) Patent Application Publication
(10) Pub. No.: US 2005/0249764 A1
(43) Pub. Date: Nov. 10, 2005

(54) Cosmetic composition containing a metal oxide and a heterogeneous polyholoside

(75) Inventor: Philippe Touzan, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/117,471

(22) Filed: Apr. 29, 2005

Related U.S. Application Data
(60) Provisional application No. 60/573,342, filed on May 24, 2004.

(30) Foreign Application Priority Data
May 7, 2004 (FR) 0450881

(51) Int. Cl. A61K 7/06; A61K 7/11

(52) U.S. Cl. 424/401; 424/70.13

(57) ABSTRACT

A composition containing, in a physiologically acceptable medium, at least one heterogeneous polyholoside and corundum particles, useful to attenuate the visible or tactile irregularities on the surface of the skin.

### REFERENCE TO PRIOR APPLICATIONS

This application claims priority to U.S. provisional application 60/573,342 filed May 24, 2004, and to French patent application 0450881 filed May 7, 2004, both incorporated herein by reference.

### FIELD OF THE INVENTION

The present invention relates to a composition comprising, preferably in a physiologically acceptable medium, at least one heterogeneous polyholoside and corundum particles.

The invention also relates to a cosmetic skincare process or “microdermabrasion” process, for example to attenuate the visible or tactile irregularities on the surface of the skin, in particular to attenuate wrinkles and fine lines and/or skin marks and/or the appearance of blackheads and/or skin pores and/or to smooth out the skin and/or to unify the complexion, comprising:
the topical application of the abovementioned composition to skin, for example by massaging into premoistened skin, and
rinsing of the composition with water.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human skin consists of two compartments, namely a surface compartment, the epidermis, and a deep compartment, the dermis.

Natural human epidermis is composed mainly of three types of cell: the keratinocytes, which form the vast majority, the melanocytes and the Langerhans cells. Each of these cell types contributes, via its intrinsic functions, towards the essential role played by the skin in the body.

During the keratinization process, the keratinocytes located in the basal layer of the epidermis multiply and grow, thus pushing the older epidermal cells upwards and towards the surface of the epidermis. During this displacement, the cells become flattened and differentiate to form keratin. The superficial dead cells resulting from this keratinization process (corneocytes) constitute the horny layer of the epidermis, where they are separated by lipid layers and linked together via protein bonds (corneosomes). These dead cells are gradually removed from the surface of the skin and replaced with new keratinized cells.

In young and healthy skin, the desquamation of the skin that takes place in this way is characterized by the removal of individual cells or of small lumps of cells. In contrast, with age or in the case of certain pathologies, the desquamation may be impaired, in the sense that an excess of keratinous material forms at the surface of the skin, resulting either in removal of the stratum corneum in the form of squamae (ageing of the skin, dry skin), or in an obstruction of the sebaceous follicles (acne).

Various agents for promoting desquamation, i.e. the removal of the “dead” cells lying at the surface of the horny layer of the epidermis, are known in the prior art. This “desquamating” property is also referred to, often incorrectly, as a keratolytic property.

Thus, patent U.S. Pat. No. 4,603,146 describes the use of retinoic acid and derivatives thereof in cosmetic compositions, for combating ageing of the skin. Moreover, many patents and publications (see, for example, patent application EP-A-413 528) and many commercial cosmetic compositions teach the use of α-hydroxy acids, for instance lactic acid, glycolic acid or citric acid, for treating ageing of the skin. Finally, β-hydroxy acids and more especially salicylic acid and derivatives thereof are known for their desquamating properties (see documents WO-93/10756 and U.S. Pat. No. 4,767,750).

Along with these “chemical” desquamating agents, it is known practice to use abrasive agents such as aluminium oxide in the form of corundum to exfoliate the surface of the skin (U.S. Pat. No. 6,290,976; WO 02/072042). This exfoliation process is known as “microdermabrasion”. Microdermabrasion is especially performed by beauticians or dermatologists, who use microcrystals of aluminium oxide (in corundum form) to resurface the upper layers of the skin. These crystals are sprayed onto the skin using a machine, which then sucks them off with the abraded skin.

Although their efficacy is satisfactory, all these prior-art desquamating and abrasive compounds can give rise to irritation or discomfort, in particular in the case of individuals with sensitive skin.

In particular, the size of abrasive agents is generally chosen as sufficient so as not to obstruct the pores of the skin and to run the risk of producing an infection or inflammation therein, but abrasive agents larger than 100 μm in size generally have a tendency to be more irritant to the skin than those of smaller size.

There is thus still a need for an abrasive composition that is well tolerated while at the same time being effective.

### SUMMARY OF THE INVENTION

The inventor has now discovered that by combining certain polyholosides with corundum particles, it is possible to mask the hard and cutting nature of this metal oxide and thus to obtain a composition that has acceptable tolerance and skin comfort, while at the same time conserving good abrasive efficacy. It is thus now possible to formulate compositions that may be used at home, with no risk for the user, or which may be used in a salon without the need for specific equipment.

Some of these polyholosides, in particular alginates, have already been used in masks (U.S. Pat. No. 2,996,432; EP-0 045 493; JP-2003 089 615; JP-2002 241 220) on account of their capacity to form a rigid gel in the presence of metal salts. In patent application EP-0 045 493, it is suggested to combine them especially with aluminium oxide. However, it is a matter therein of a compound used to modify the texture or the stability of the gel formed by the alginate, and therefore quite probably aluminium oxide in amorphous alumina form. Amorphous alumina forms at about 50-60° C. and is commonly used as a colourless filler when it is in hydrated form. It thus differs from corundum, which is generally obtained by electrofusion of alumina calcined at high temperature (above 350° C.), and which corresponds to alpha alumina of hexagonal form, commonly used as an abrasive agent.

Other polyholosides, based on fucose, have moreover been described as desquamating agents (EP-0 818 200). However, it has never been suggested to combine them with corundum particles, in particular for a use in a microdermabrasion process.

### DETAILED DESCRIPTION OF THE PREFERRED EMBODIMENTS

One subject of the present invention is thus a composition comprising, preferably in a physiologically acceptable medium, at least one heterogeneous polyholoside and corundum particles.

The polyholoside not only has the advantage of reducing the irritation resulting from the abrasive nature of corundum, but also of complementing this abrasive, mechanical action with a “biological” exfoliating action, by inducing the release of corneocytes, as demonstrated in patent application EP-0 818 200.

These two constituents of the composition according to the invention will now be described in greater detail.

Heterogeneous Polyholoside

According to the present invention, the term “heterogeneous polyholoside” means polymers having a combination of different saccharides or of saccharides having the same empirical chemical formula but different geometrical configurations (for example D and L isomers) and preferably are polymers consisting of such combinations and mixtures thereof.

These polymers differ both from polyheterosides, which consist of one or more saccharides and of a non-carbohydrate portion, and from homogeneous polyholosides, which result from the combination of the same saccharide.

Thus, the heterogeneous polyholoside according to the invention preferably consists solely of saccharides and results from the combination of at least two different saccharides or of saccharides having the same empirical chemical formula but different geometrical configurations.

The polyholosides according to the invention may preferably consist of 2 to 10 saccharides, these compounds commonly being referred to as oligoholosides, or of more than 10 saccharides, these compounds commonly being referred to as polyholosides.

The saccharides present in the polyholoside according to the invention may be chosen from any saccharide of natural or synthetic origin that may be known or envisaged, and especially such as:
aldoses, for instance
   pentoses: ribose, arabinose, xylose or apiose, for example,
   hexoses: glucose, fucose, mannose or galactose, for example,
ketoses such as fructose,
deoxyoses, such as rhamnose, digitoxose, cymarose or oleandrose,
saccharide derivatives such as uronic acids, for instance mannuronic, guluronic, galacturonic or glycuronic acids, or itols, for instance mannitol or sorbitol.

In the context of the present invention, a heterogeneous polyholoside alone or a mixture of heterogeneous polyholosides may be used.

The polyholoside according to the invention may be branched or linear. It may also be substituted, for example with fatty chains, especially preferably containing 8 to 30 carbon atoms.

Moreover, the polyholoside according to the invention may be an alginate (poly mannuronate and guluronate) such as a sodium alginate, a propylene glycol alginate, a calcium alginate or a glyceryl alginate.

However, the heterogeneous polyholoside preferably comprises at least one fucose unit, which may be present in an amount of 10-90% by weight and preferably 15-35% by weight relative to the weight of polyholoside solids.

In particular preferred embodiments, the polyholoside according to the invention may comprise fucose, galactose and galacturonic acid units, and, for example, may comprise a linear sequence of α-L-fucose, α-D-galactose and galacturonic acid. In this case, it preferably has a viscosity of 800-1200 mPa·s (Brookfield LV31 viscosity, 12 rpm, at 30° C.) when it is dissolved in water to a concentration of about 1% by weight. Such a polyholoside is especially available in the form of a 1% solution in water from the company Solabia under the trade name Fucogel 1000 PP®.

The polyholosides according to the invention are preferably introduced into the composition in the form of an aqueous solution that may comprise 0.1% to 5% by weight of polyholoside.

The polyholoside may for example be present in the composition according to the invention in an amount representing from 0.0001% to 1% by weight and preferably from 0.0005% to 0.01% by weight relative to the total weight of the composition.

Corundum

The corundum that may be used according to the invention is an aluminium oxide, which may be coated, for example, especially with a silicone compound, but is preferably uncoated.

The corundum particles advantageously have a purity of at least 95% and better still of at least 99%.

Their mean particle size preferably ranges from 100 to 180 μm.

The corundum particles that may be used according to the invention are in particular aluminium oxide particles calcined at high temperature, until the crystalline structure of corundum (α-Al₂O₃) is obtained, and then treated to form grains having cutting edges and having a given particle size distribution, the particles preferably having a mean particle diameter of between 100 and 180 μm and preferably between 130 and 150 μm. Their distribution is advantageously such that none of the particles has a diameter of greater than 250 μm. Such particles are especially commercially available from the company MarkeTech International under the trade name Dermagrain. The particles referenced Dermagrain 900 consist of 99.55% pure crystalline alpha-alumina, with a mean particle diameter of about 140 μm, the particles all having a diameter of less than 250 μm. Less than 3% of the particles have a diameter of less than 105 μm. Other particles are available from the company Industrial Supply under the trade name ARL 100 and ARL 120. They are aluminium oxide particles with a mean particle size of 120 and 100 μm, respectively, and a particle size distribution ranging from 75 to 212 μm and from 63 to 180 μm, respectively.

The corundum particles may represent for example from 5% to 40% and preferably from 10% to 30% of the total weight of the composition.

The composition according to the invention is generally preferably adapted for topical application to the skin and thus generally comprises a physiologically acceptable medium, i.e. a medium that is compatible with the skin and/or its integuments. Thus, the composition according to the invention preferably has a pH of less than 8, better still less than or equal to 7 and even better still between 5.5 and 7.

The composition according to the invention may be in any galenical form, including those conventionally used for topical application, preferably such that it can be rinsed off with water, and especially in the form of dispersions of the lotion or gel type or emulsions of liquid or semi-liquid consistency of the milk type, obtained by dispersing a fatty phase in an aqueous phase (O/W), or suspensions or emulsions of soft, semi-solid or solid consistency of the cream or gel type, obtained by dispersing a fatty phase in an aqueous phase (O/W), or alternatively multiple emulsions (W/O/W), microemulsions, vesicular dispersions of ionic and/or nonionic type, or wax/aqueous phase dispersions. These compositions can be prepared according to the usual methods.

According to one preferred embodiment of the invention, the composition is in the form of an oil-in-water (O/W) emulsion.

As oils that may be used in the composition according to the invention, mention may be made of:
hydrocarbon-based oils of animal origin, such as perhydrosqualene;
hydrocarbon-based oils of plant origin, such as plant oils, in particular the liquid fraction of shea butter;
synthetic esters and ethers, especially of fatty acids, for instance pentaerythritol esters such as pentaerythrityl tetraethylhexanoate;
linear or branched hydrocarbons of mineral or synthetic origin, such as hydrogenated polyisobutene and mineral oil;
fatty alcohols containing from 8 to 26 carbon atoms, for instance cetyl alcohol, stearyl alcohol and the mixture thereof, or octyldodecanol;
silicone oils, for instance volatile or non-volatile polymethylsiloxanes (PDMS) containing a linear or cyclic silicone chain; and
mixtures thereof.

When it is present, the fatty phase of the composition according to the invention may comprise, besides oils, other fatty substances such as: fatty acids containing from 8 to 30 carbon atoms; waxes; and gums such as silicone gums (dimethiconol).

These fatty substances may be chosen in a varied manner by a person skilled in the art so as to prepare a composition having the desired properties, for example in terms of consistency or texture.

This composition may also contain various adjuvants such as those commonly used in cosmetics, such as emulsifiers including glyceryl stearate and poly(ethylene oxide) stearates; fillers; preserving agents; sequestering agents; dyes; fragrances; and thickeners and gelling agents, in particular acrylamide homopolymers and copolymers, and 2-acrylamido-2-methyl-propanesulfonic acid homopolymers and copolymers, which are optionally crosslinked and/or neutralized, for instance the poly(2-acrylamido-2-methylpropanesulfonic acid) sold by the company Clariant under the name “Hostacerin AMPS”. The amounts of these various adjuvants and their nature preferably are chosen so as not to harm the properties of the composition according to the invention.

To reinforce the effects of the composition according to the invention, it may moreover contain at least one compound chosen from: moisturizers such as ceramides, glycerol and hyaluronic acid; desquamating agents such as α-hydroxy acids, including glycolic and lactic acids, β-hydroxy acids, in particular 5-n-octanoylsalicylic acid, HEPES or honey; antiseborrhoeic agents such as zinc salts or certain extracts of kelps or of meadowsweet; calmatives such as β-glycyrrhetinic acid and its salts and/or its derivatives and extracts of peony or of aloe vera; and mixtures thereof.

The composition according to the invention may be applied to the skin by any method such as manual massaging with the fingertips, by mechanical massaging using a vibrating machine equipped with a massaging head fitted with a pad, as described in patent application U.S. 2001/0 046 506 or patent U.S. Pat. No. 6,652,888, for example, etc.

The composition according to the invention allows the visible and tactile irregularities on the surface of the skin to be effaced, by virtue of its capacity to mechanically abrade the surface of the skin, but also to accelerate cell renewal.

A subject of the invention is thus also a cosmetic skincare process or a “microdermabrasion” process for attenuating the visible or tactile irregularities on the surface of the skin, in particular for attenuating wrinkles and fine lines and/or skin marks and/or the appearance of blackheads and/or skin pores and/or for smoothing out the skin and/or for unifying the complexion, comprising:
the topical application of the abovementioned composition to skin, for example by massaging into premoistened skin, and
rinsing of the composition with water.

The frequency with which this process may be performed will depend on the desired result. For example it can generally be performed one to three times a week, preferably twice a week, for a period ranging from one week to six months and preferably from four to eight weeks.

In addition, the process according to the invention may comprise an additional step of applying a moisturizing and/or calmative composition to the skin, as well as other steps.

This process is advantageously performed on individuals presenting signs of ageing of the skin, such as wrinkles and fine lines and/or pigmentation irregularities and/or a dull complexion, or on individuals with dilated pores and/or blackheads.

The invention will now be illustrated by means of the non-limiting examples that follow. In these examples, the amounts are indicated as weight percentages.

### EXAMPLES

### Example 1

### Microdermabrasion Cream

An O/W emulsion having the composition below is prepared, in a standard manner for a person skilled in the art.

| | | |
|---|---|---|
| Phase A | PEG stearate (100 EO) | 0.75% |
| | glyceryl stearate | 0.75% |
| | stearyl alcohol | 1.5% |
| | mineral oil | 10% |
| | apricot kernel oil | 2% |
| | polydimethylsiloxane | 1% |
| Phase B | demineralized water | qs 100% |
| | 1% polyholoside* | 1% |
| | polyacrylamide | 0.3% |
| | butylene glycol | 3% |
| | propylene glycol | 3% |
| Phase C | corundum** | 15% |
| Phase D | demineralized water | 5% |
| | preserving agents | qs |

*Fucogel 1000 PP from Solabia
**Dermagrain 900 from MarkeTech International

This cream may be applied in the evening to the moistened face, avoiding the contour of the eyes and the lips, by massaging with the fingertips for about one and a half minutes, followed by rinsing with water. It makes it possible to improve the radiance of the complexion, smooth out the skin surface and reduce the appearance of wrinkles.

### Example 2

### Cleansing Foaming Gel

The composition below is prepared in a standard manner for a person skilled in the art:

| Phase A | demineralized water | qs 100% |
|---|---|---|
| | EDTA | 0.2% |
| | preserving agent | qs |
| | acrylic copolymer (Pemulen TR1) | 1% |
| | sodium lauroyl sarcosinate | 2% |
| | sodium laureth sulfate | 5% |
| Phase B | demineralized water | 10% |
| | 1% polyholoside* | 2% |
| | xanthan gum | 0.3% |
| | butylene glycol | 3% |
| | propylene glycol | 3% |
| | preserving agent | qs |
| | triethanolamine | 0.8% |
| | corundum** | 6% |

*Fucogel 1000 PP from Solabia
**Dermagrain 900 from MarkeTech International

This gel may be applied in the morning or evening to the moistened face, avoiding the contour of the eyes and the lips, by massaging with the fingertips for about one and a half minutes, followed by rinsing with water. It makes it possible to reduce the appearance of blackheads and to close up skin pores.

### Example 3

### In Vivo Assay

Principal:

Two compositions A and B, each consisting of a gelled oil-in-water emulsion containing 20% by weight of corundum particles (Dermagrain 900 from MarkeTech International), composition B also containing 0.2% by weight of heterogeneous polysaccharide at 1% by weight in water (Fucogel 1000 PP from Solabia), were tested in a half-face test on a panel of 10 individuals.

Protocol:

After moistening the face with a sponge, the beautician applied 1 g of product to half the face, followed by gentle massaging and then removal of the product by rinsing with a sponge. The face was then dried by dabbing with a handkerchief.

Result:

For 4 out of 10 individuals, composition A is perceived as being more abrasive than composition B, in the sense that the grains appear to be coarser, harder and more perceptible than on the other half of the face treated using composition B. In addition, 7 out of the 10 individuals discontinued the application of composition A on account of this discomfort, judging composition B to be more comfortable, whereas only 3 out of 10 individuals discontinued the application of composition B.

In addition, after applying compositions A and B, the skin appears fresh, clean and softer and smoother than before the application.

These results thus show that composition B is perceived as being just as effective, but markedly less abrasive than, composition A and therefore better tolerated.

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, at least one heterogeneous polyholoside and corundum particles.

As used above, the phrases “selected from the group consisting of,” “chosen from,” and the like include mixtures of the specified materials. The term “contain” and its variants means “comprise.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium, at least one heterogeneous polyholoside and corundum particles.

2. The composition according to claim 1, comprising an alginate heterogeneous polyholoside.

3. The composition according to claim 1, comprising at least one heterogeneous polyholoside that comprises at least one fucose unit.

4. The composition according to claim 3, wherein said at least one heterogeneous polyholoside that comprises at least one fucose unit comprises fucose, galactose and galacturonic acid units.

5. The composition according to claim 1, wherein the at least one heterogeneous polyholoside represents from 0.0005% to 0.01% by weight relative to the total weight of the composition.

6. The composition according to claim 1, wherein the mean size of the corundum particles ranges from 100 to 180 μm.

7. The composition according to claim 6, wherein the corundum particles have a mean particle diameter of 130-150 μm.

8. The composition according to claim 6, wherein none of the corundum particles has a diameter of greater than 250 μm.

9. The composition according to claim 1, wherein the corundum particles represent from 5% to 40% of the total weight of the composition.

10. The composition according to claim 9, wherein the corundum particles represent from 10% to 30% of the total weight of the composition.

11. The composition according to claim 1, wherein said composition has a pH of 5.5-7.

12. The composition according to claim 1, wherein said composition in the form of an oil-in-water emulsion.

13. The composition according to claim 4, wherein said at least one heterogeneous polyholoside that comprises at least one fucose unit comprises a linear sequence of α-L-fucose, α-D-galactose and galacturonic acid.

14. A process for attenuating visible or tactile irregularities on the surface of the skin, comprising:
topically applying the composition according to claim 1 into the premoistened skin by massaging, and
rinsing the composition from the skin with water.

15. The process according to claim 14, wherein said process is a process for attenuating wrinkles and fine lines and/or skin marks and/or the appearance of blackheads and/or skin pores and/or for smoothing out the skin and/or for unifying the complexion, and wherein said composition is topically applied to skin in need of such treatment.

16. The process according to claim 14, wherein said process is repeated one to three times a week.

17. The process according to claim 14, wherein said process is a process for treating wrinkles and fine lines and/or pigmentation irregularities and/or a dull complexion, and/or dilated pores and/or blackheads and wherein said composition is topically applied to skin in need of such treatment.

18. The composition according to claim 1, wherein said corundum particles are coated.

19. The composition according to claim 1, wherein said corundum particles are coated with a silicone compound.

20. The composition according to claim 1, wherein said corundum particles are 99.55% pure crystalline alpha-alumina, with a mean particle diameter of about 140 μm, the particles all having a diameter of less than 250 μm, less than 3% of the particles having a diameter of less than 105 μm.
